(19) United States
(12) Patent Application Publication
(10) Pub. No.: US 2026/0346041 A1
(43) Pub. Date: Nov. 5, 2026

(54) BATTERY POWERED SYSTEMS FOR LIGHT THERAPY AND RELATED METHODS

(71) Applicant: BioPhotas, Inc., Tustin, CA (US)

(72) Inventors: Patrick Lamberth Johnson, Santa Ana, CA (US); Roger Allen Gibson, Lake Forest, CA (US)

(21) Appl. No.: 19/547,038

(22) Filed: Feb. 23, 2026

Related U.S. Application Data
(63) Continuation of application No. 18/118,988, filed on Mar. 8, 2023, now Pat. No. 12,558,562.
Application No. 18/118,988 is a continuation-in-part of application No. 17/162,966, filed on Jan. 29, 2021, now Pat. No. 12,447,353.
Application No. 17/162,966 is a continuation of application No. 15/980,277, filed on May 15, 2018, now abandoned.
Application No. 15/980,277 is a continuation of application No. 14/534,083, filed on Nov. 5, 2014, now Pat. No. 9,968,799.
Application No. 14/534,083 is a continuation of application No. 13/672,554, filed on Nov. 8, 2012, now Pat. No. 8,900,283.
(63) Continuation of application No. 17/187,370, filed on Feb. 26, 2021, now abandoned.
(60) Provisional application No. 61/557,319, filed on Nov. 8, 2011.
(60) Provisional application No. 62/983,247, filed on Feb. 28, 2020.

(51) Int. Cl. A61N 5/06

(52) U.S. Cl. CPC A61N 5/06; A61N 5/0613; A61N 5/0616; A61N 5/0601; A61N 2005/0626; A61N 2005/0632; A61N 2005/0642; A61N 2005/0643; A61N 2005/0645; A61N 2005/0652; A61N 2005/0659; A61N 2005/0663

(57) ABSTRACT

Battery powered light therapy systems capable of delivering 2-10 Joules per cm. sq. of light energy to a target treatment zone covering more than 700 sq. cm. of body surface area such that the light penetrates to a depth of 2 to 8 mm below a skin surface of the target treatment zone.

### RELATED APPLICATIONS

This is a continuation of U.S. patent application Ser. No. 18/118,988 entitled Battery Powered Systems for Light Therapy and Related Methods filed on Mar. 8, 2023 and issuing on Feb. 24, 2026 as U.S. Pat. No. 12,558,562, which is a) a continuation-in-part of copending U.S. patent application Ser. No. 17/162,966 entitled Shapeable Light Therapy Device and Method filed Jan. 29, 2021, which is a continuation of U.S. patent application Ser. No. 15/980,277 filed May 15, 2018 and now abandoned, which is a continuation of U.S. patent application Ser. No. 14/534,083 entitled Shapeable Light Therapy Device and Method filed Nov. 5, 2014 and issued May 15, 2018 as U.S. Pat. No. 9,968,799, which is a continuation of U.S. patent application Ser. No. 13/672,554 entitled Shapeable Light Therapy Device and Method filed Nov. 8, 2012 and issued Dec. 2, 2014 as U.S. Pat. No. 8,900,283, which claims priority to U.S. Provisional patent Application No. 61/557,319 entitled Shapeable Light Therapy Device and Method filed Nov. 8, 2011 and b) a continuation of copending U.S. patent application Ser. No. 17/187,370 entitled Battery Powered Systems for Light Therapy and Related Methods filed Feb. 26, 2021 which claims priority to U.S. Provisional Patent application No. 62/983,247 entitled Battery Powered Systems for Light Therapy and Related Methods filed Feb. 28, 2020, the entire disclosure of each such patent and application being expressly incorporated herein by reference.

### FIELD OF THE INVENTION

The present invention relates generally to the fields of physics, electronics, biology and medicine and more particularly to devices and methods for delivering light therapy to humans or animals.

### BACKGROUND

Pursuant to 37 CFR 1.71(e), this patent document contains material which is subject to copyright protection and the owner of this patent document reserves all copyright rights whatsoever.

Light therapy (i.e., “phototherapy”), using various types of light, has been used or proposed for use in a number of cosmetic and therapeutic applications, including but not necessarily limited to improvement of skin elasticity, deterrence of skin aging, treatment of dermatological disorders (e.g., acne, psoriasis), healing of wounds, treatment of jaundice in newborns, and treatment of certain psychological conditions such as seasonal affective disorder (SAD) and certain sleep disorders. In some applications, light therapy is used alone while in others it is used in combination with drugs or agents (e.g., photo-sensitizing agents, photo-activating agents, agents which reduce skin opacity or improve light penetration through or into the skin, etc.).

Applicant is the owner of related U.S. Pat. No. 8,900,283 (Johnson) and U.S. Pat. No. 9,968,799 (Johnson), the entire disclosures of which are expressly incorporated herein by reference.

### SUMMARY

The present invention provides new light therapy systems and methods wherein a light emitting device comprising a light emitting pad member is sized and configured to extend over at least part of a subject's body. In some embodiments the light emitting pad may be configured to extend over all, substantially all or more than half of the subject's body.

In accordance with one aspect of the present disclosure, there is provided a light therapy system useable for delivery of light therapy. This system may comprise: a light emitting pad member having light emitter(s) configured to emit blue light and/or red light and/or near infrared light and a battery. The pad member may be formable into a shape which corresponds to a shape of a body portion so as to be positionable on or near that body portion with said light emitter being within a desired distance of a target treatment zone located on or in that body portion. The battery delivers power to the light emitter(s). Such battery, and the desired distance at which the pad is positioned, may be configured such that the battery will power the light emitter(s) sufficiently to cause the light emitter(s) to emit light which travels said desired distance and delivers 2-10 Joules per square centimeter of light energy within the target treatment zone.

Further in accordance with the present disclosure, the tight therapy system may be configured such that the battery delivers sufficient power to the light emitter(s) to cause the light emitter(s) to deliver 2 to 10 Joules per cm. sq. of light energy to a penetration depth of up to 25 mm below a skin surface covering a target treatment zone. The target treatment zone may cover an area that exceeds: 100 square centimeters, or 200 square centimeters, or 300 square centimeters, or 400 square centimeters, or 500 square centimeters, or 600 square centimeters, or 700 square centimeters of body surface area, or the size of the target treatment zone may vary (e.g., by using pads of differing size) within a range defined by any two of the preceding values. In one embodiment the pad member and light emitter(s) are configured to deliver light to a target treatment zone that covers 718.5 square centimeters of skin or body surface area.

Further in accordance with the present disclosure, the light emitter(s) may comprise red LEDs and the system may be configured such that the battery delivers sufficient power to the red LEDs to cause the red LEDs to deliver 2-10 Joules per cm. sq. of red light to a target treatment zone that covers more than 700 sq. cm. of body surface area such that the red light penetrates to a depth of 2 to 8 mm below a skin surface of the target treatment zone.

Still further in accordance with the disclosure, there are provided methods for using the above-summarized light therapy systems to deliver light therapy to the bodies of humans or other animals.

Additional aspects and details of the light therapy system and associated methods will be understood upon reading of the detailed description and examples set forth herebelow.

### BRIEF DESCRIPTION OF THE DRAWINGS

The following figures are included in this provisional patent application and referenced in the following description and claim statements. These figures are intended only to illustrate certain aspects or embodiments of the disclosed system and are not limiting in any respect.

FIG. 1 is a schematic diagram showing one embodiment of a system of the present disclosure.

FIG. 1A is a schematic diagram showing an example of a target treatment zone for blue light therapy in accordance with the present disclosure.

FIG. 1B is a schematic diagram showing an example of a target treatment zone for red light therapy in accordance with the present disclosure.

FIG. 1C is a schematic diagram showing an example of a target treatment zone for near infrared light therapy in accordance with the present disclosure.

FIG. 2 is a bottom view of a controller/user interface in accordance with the present disclosure.

FIG. 3 is a front view of a controller/user interface in accordance with the present disclosure.

FIG. 4 is a left side view of a controller/user interface in accordance with the present disclosure.

FIG. 5 is a right side view of a controller/user interface in accordance with the present disclosure.

FIG. 6 is a top view of a controller/user interface in accordance with the present disclosure.

FIG. 7 shows an embodiment of a light therapy system with an exploded view of a controller/user interface component of the system.

FIG. 8 is an electrical block diagram of a light therapy system according to the present disclosure.

FIG. 9 is a detailed block diagram of a charge board component of a light therapy system according to the present disclosure.

FIG. 10 is an electrical schematic showing power input and battery portions of a charge board component of a light therapy system according to the present disclosure.

FIG. 11 is an electrical schematic showing boost regulation and output portions of a charge board component of a light therapy system according to the present disclosure.

### DETAILED DESCRIPTION

As used in this provisional patent application the term “pad member” is to be interpreted broadly and shall include any suitable structure including, but not necessarily limited to, flexible flat or planar structures, pads, mats, panels, sheets, blankets, etc. Light emitters, such as light emitting diodes (LEDs), emit light from one side (i.e., a light-emitting side) of the pad. The pad may be positioned under or over the body of a subject such that light which emanates from the light-emitting side of the pad is cast on the subject body thereby providing light therapy. Optionally, the pad may be flexible and one or more shapeable member(s) may be positioned on or in region(s) of the pad to render such region(s) of the pad formable into shape(s) which conform to a body part of the subject or which facilitate placement on or in abutment with an underlying or adjacent surface.

The following detailed description and the accompanying drawings to which it refers are intended to describe some, but not necessarily all, examples or embodiments of the invention. The described embodiments are to be considered in all respects only as illustrative and not restrictive. The contents of this detailed description and the accompanying drawings do not limit the scope of the invention in any way.

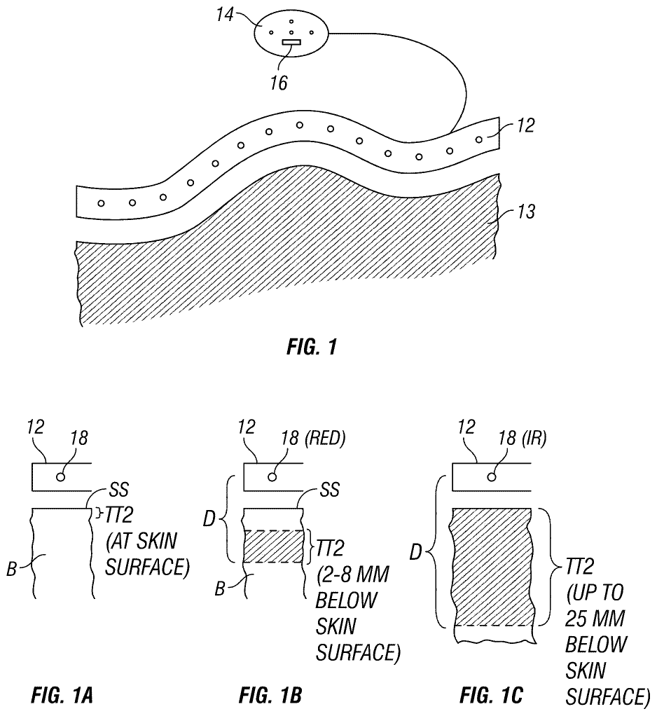

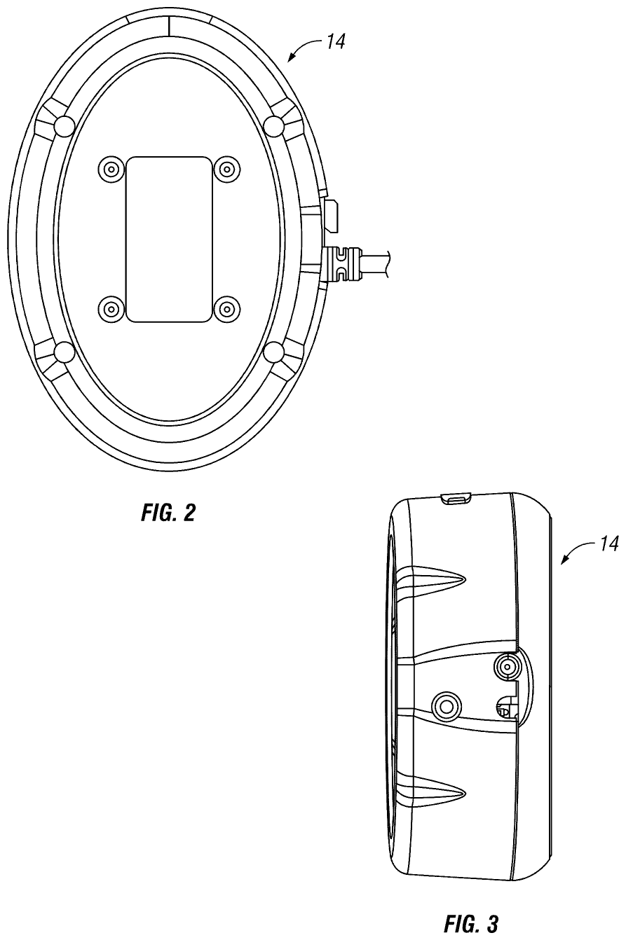

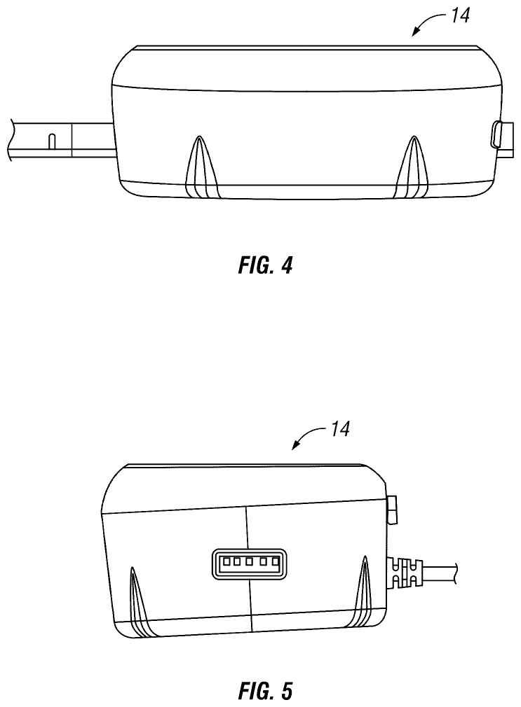

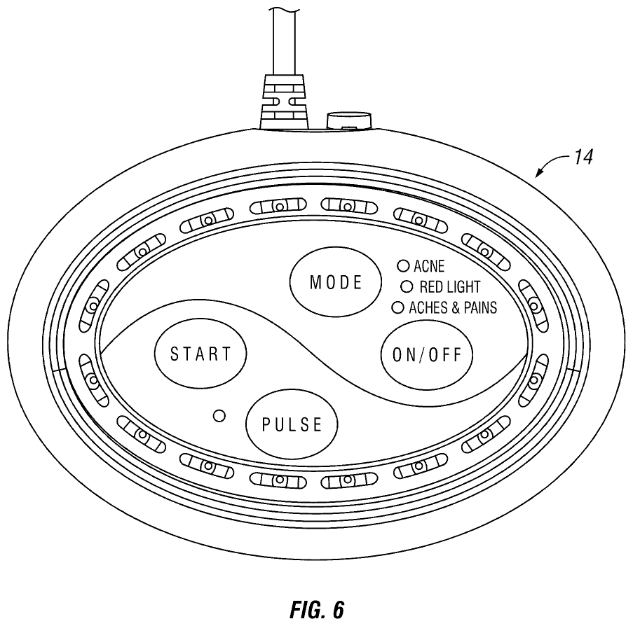

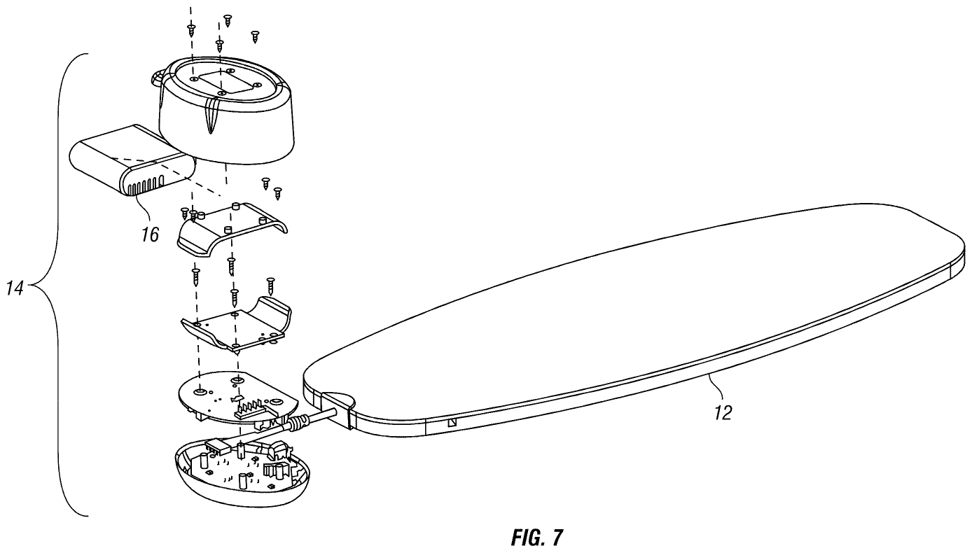

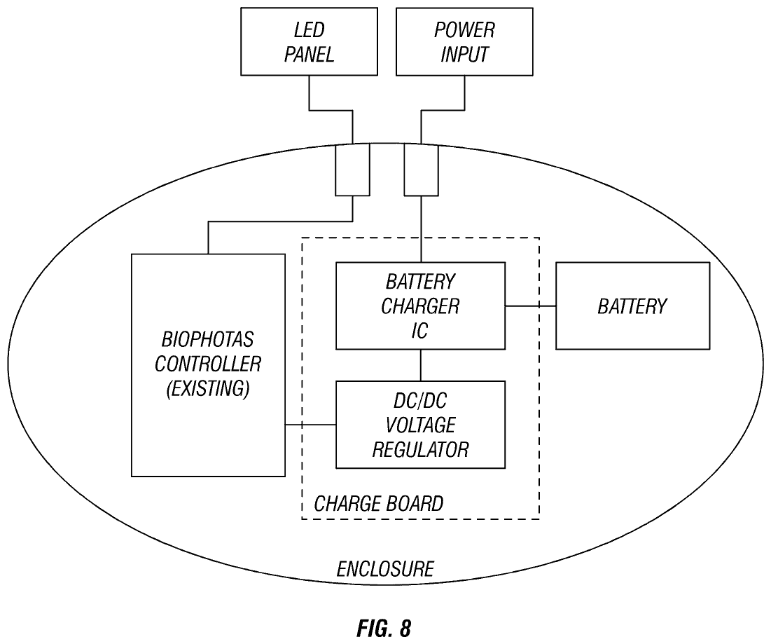

FIG. 1 is a schematic diagram of a system 10 of the present invention positioned to provide light therapy to a portion of a human body B. This system 10 includes at least one shapeable light emitting pad 12 having light emitters 18 and a controller/user interface 14 and a battery 16. The battery 16 may be housed within the controller/user interface 14. The pad 12 is preferably formable into a plurality of retained shapes so that the shape of the pad 12 may correspond to various curved body surfaces so that the light emitters 18 are within a desired distance D of a target treatment zone TTZ such that the light which reaches the target treatment zone TTZ is of sufficient intensity to cause increased uptake of ATP in cells within the target treatment zone TTZ. Clinical data indicates that delivering 2-10 Joules per cm. sq. will trigger the up-regulation of ATP at a particular target treatment zone TTZ. The following Table 1 below and FIGS. 1A, 1B and 1C show target treatment zones TTZs for red, blue and near infrared LED light emitters 18.

| | | Target Treatment Zone |
|---|---|---|
| Light | Wavelength (nM) | (TTZ) |
| Blue | at or approximately 460 | At the skin surface |
| Red | 640 | 2 to 8 mm below the skin |
| | | surface |
| Near | 880 | Up to 25 mm below the |
| infrared | | skin surface. |

The wavelengths shown in Table 1 may vary. For example, such wavelengths bay vary+/−5 nM. Alternatively, such wavelengths may vary+/=10 nM. Alternatively, such wavelengths may be “approximate,” meaning that the specified wavelength may vary by any amount that does not render the light ineffective for its stated therapeutic purpose.

The battery 16 is capable of providing sufficient power to cause each light emitter 18 to emit light from the pad 12, over the desired distance D so that light energy of 2-10 Joules per square centimeter of will reach the target treatment zone TTZ for that type of light emitter 18. The delivery of this dosage of light therapy results in upregulation of ATP in cells within that target treatment zone TTZ. A battery useable in this system may draw<200 uA in standby Minimum Voltage 4.8V Charge Voltage 8.438V Max Charge Current 2.046 A Capacity>=6120 mAh Impedance<206 mOhms fully charged Blade connector is not keyed. Typical pinout has Pin 1 as battery positive.

As illustrated in FIG. 1A, for light emitters 18 which emit blue light, the battery 16 will provide sufficient power to cause blue light to travel a distance D from the blue light emitters 18 to a target treatment zone TTZ located at the skin surface SS such that 2 to 10 Joules per cm. sq. of blue light energy is delivered within that target treatment zone TTZ (the skin surface).

As illustrated in FIG. 1B, for light emitters 18 that emit red light, the battery 16 will provide sufficient power to cause red light to travel a desired distance D from the red light emitters 18 to a target treatment zone TTZ that is 2 to 8 mm below the skin surface such that 2-10 Joules per cm. sq. of red light energy is delivered within that target treatment zone TTZ (2 to 8 mm below the skin surface.)

As illustrated in FIG. 1C, for light emitters that emit near infrared light, the battery 16 will provide sufficient power to cause near infrared light to travel a desired distance D from the near infrared light emitters to the target treatment zone (up to 25 mm below the skin surface) such that 2-10 Joules per cm. sq. of near infrared light energy is delivered within that target treatment zone TTZ (up to 25 mm below the skin surface).

One type of battery that is suitable for use in a system 10 that includes blue, red and near infrared light emitters 18 is a 4 cell, 7.2 volt, 6.8 Amp hour lithium ion battery. The voltage is stepped up to 12V.

In some embodiments the pad 12 may be place in contact with the skin surface, while in other embodiments, the pad 12 may remain a spaced distance away from the skin surface. In either event the light emitters 18 will be within the desired distance D of the target treatment zone TTZ for that type of light emitter 18. The pad 12 may be formable into, and will retain without a strap or other retaining member, a plurality of different shapes, each of which has a plurality of curves (i.e., “multi-curvate” shapes). In this manner, the pad 12 may be pre-shaped to conform to the configuration of a body portion to the treated so that, when the pad 12 is placed on or near that body portion, all of substantially all of the pad's light emitters 18 will be within the desired distance D of the target treatment zone TTZ.

In embodiments where a portion of the pad 12 directly contacts the skin surface SS, the skin-contacting portion of the pad 12 may comprise a translucent panel through which the light is cast from the light emitters 18. Such skin-contacting translucent panel may be formed of material that can be sanitized after each use. Alternatively, such translucent panel may be covered with a disposable barrier layer, such as a clear plastic film, that may be peeled away and discarded after each use. In such embodiments, the light emitters 18 may be positioned within the pad 12 so that, when the translucent panel is in contact with the subject's skin, the light emitters 18 will be nominally ⅓ of a centimeter from the surface of the skin. This close emission proximity is designed to leverage the Inverse Square Law that states that as the distance between a light emitter 18 and a surface of absorption (the skin) is doubled the energy available for absorption decreases by 4 times. In application, this means the closer to the skin surface SS the light emitters 18 are positioned, the less power required from the battery 16 to deliver a desired therapeutic dose of light energy (e.g., 2-10 Joules per cm. sq.) to the target treatment zone TTZ. Pads 12 of varied sizes may be used. The herein described system is capable of operating with a pad 12 sized to deliver 2 to 10 (e.g, up to 10) Joules per cm. sq. of light energy (e.g., red light) to a depth of 2 to 8 mm (e.g., up to 8 mm) below the skin surface over a target treatment zone TTZ covering more than 700 square centimeters (e.g., 718.5 square centimeters) of body surface area.

In some embodiments, the system 10 may be programmed to deliver light therapy in a plurality of alternative light treatment modes intended for different therapeutic or cosmetic applications, including a) one light treatment mode wherein the emitted light is primarily near infrared; b) another light treatment mode wherein the emitted light is primarily red; and c) yet another light treatment mode wherein the emitted light is primarily blue. Operation of the device 10 in the near infrared treatment mode may cause the LEDs to emit light having a wavelength of, or of about, 880 nm. Operation of the device 10 in the red treatment mode may cause the LEDs to emit light having a wavelength of, or of about, 640 nm. Operation of the device 10 in the blue treatment mode may cause the LEDs to emit light having a wavelength of, or of about, 465 nm. As explained above, these different treatment modes may be selected depending on the pathological or cosmetic condition being treated and/or the depth of light penetration desired. See, Bartolet, D., Light-Emitting Diodes (LEDs) in Dermatology; Seminars in Cutaneous Medicine and Surgery, Vol. 27: pp. 227-238 (2008).

The controller/user interface 14 may include a switch for turning the power on/off and a selector for selecting which treatment mode is desired. Also, optionally, the treatment times may be fixed or the user interface may include a timer set for setting the desired treatment time. Also, optionally, the device may be programmed to emit light in each treatment mode in either a pulsed (e.g., modulated) or non-pulsed fashion and the user interface may include a switch or function to allow the user to select or not select whether pulsing (e.g., modulation) is desired. For example, the device 10 may be sent to default to a pulsed delivery of light in each treatment mode unless the user inputs a signal through the user interface 14 to terminate the pulsing. More specifically, in this non-limiting example, when a light therapy session is initiated with the device set in the one treatment mode, the blue LEDs will emit blue light at a 1% duty cycle and the red and near infrared LEDs will fade up from 1% to 90% in 20 seconds. When a light therapy session is initiated with the device set in another treatment mode, the blue LEDs will fade up from 1% to 90% in 20 seconds and the red and near infrared LEDs will fade up from 1.3% to 2.5% in 2.5 seconds. Also, when a light therapy session is initiated with the device set in yet another treatment mode, the blue LEDs will fade up from 1% to 90% in 20 seconds and the red and near infrared LEDs will cycle from 30% to 80% in 11.5 seconds. In this particular non-limiting example, each treatment mode will deliver pulsed light unless pulsation is turned off via the user interface 14, as follows: a) the first light treatment mode will deliver light at a pulse width modulation frequency of about 680 Hz unless pulse width modulation is turned off via the user interface 14; b) the second light treatment mode delivers light at a pulse width modulation frequency of about 800 Hz unless pulse width modulation is turned off via the user interface 14 and the third light treatment mode delivers light at a pulse width modulation frequency of about 80 Hz unless pulse width modulation is turned off via the user interface 14. As explained above, this ability to select the desired modulation (e.g., pulsation or non-pulsation) allowed the system 10 to be used to achieve different therapeutic effects. See, Bartolet, D., Importance of Pulsing Illumination Parameters in Low-Level-Light Therapy; Journal of Biomedical Optics, Vol. 15, No. 4: pp. 048001-048005 (2010).

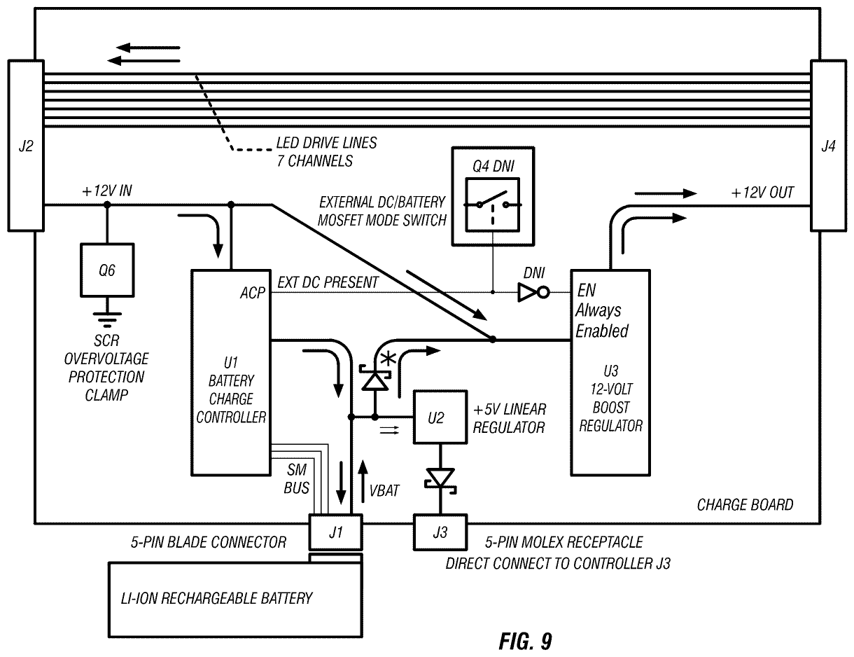

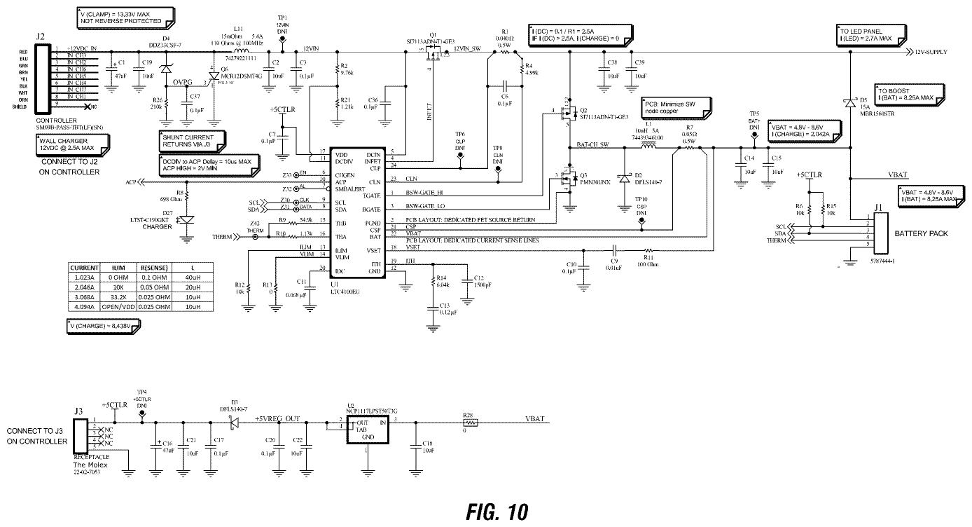

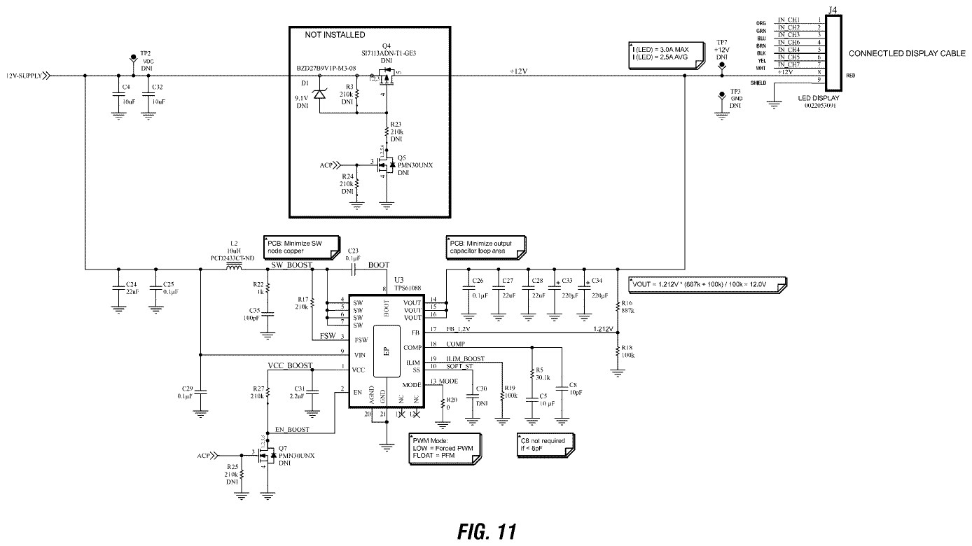

FIGS. 9 thorough 11 show additional details of a the battery controller and charge board.

In some embodiments the controller/user interface 14 may include a display. Such display may display indications of whether the power is on or off and what light treatment mode has been selected. Optionally, such display may also display a treatment time that has been selected and/or elapsed and/or remaining; and, optionally, whether pulse width modulation is on or off.

In some embodiments, the controller/user interface may also be useable to switch between a pulsed mode and a non-pulsed mode as described in the above-incorporated U.S. Pat. No. 8,900,283 (Johnson) and U.S. Pat. No. 9,968,799 (Johnson).

Although the invention has been described hereabove with reference to certain examples or embodiments of the invention, various additions, deletions, alterations and modifications may be made to those described examples and embodiments without departing from the intended spirit and scope of the invention. For example, any elements, steps, members, components, compositions, reactants, parts or portions of one embodiment or example may be incorporated into or used with another embodiment or example, unless otherwise specified or unless doing so would render that embodiment or example unsuitable for its intended use. Also, where the steps of a method or process have been described or listed in a particular order, the order of such steps may be changed unless otherwise specified or unless doing so would render the method or process unsuitable for its intended purpose. Additionally, the elements, steps, members, components, compositions, reactants, parts or portions of any invention or example described herein may optionally exist or be utilized in the absence or substantial absence of any other element, step, member, component, composition, reactant, part or portion unless otherwise noted. All reasonable additions, deletions, modifications and alterations are to be considered equivalents of the described examples and embodiments and are to be included within the scope of the following claims.

What is claimed is:

1.-20. (canceled)

21. A device for delivering light therapy to a treatment area on a human body, said device comprising:
a light therapy pad which has a back side, a front side, first and second ends and first and second lateral edges, wherein the back side of the pad comprises a layer of flexible material and the front side of the pad comprises a transparent shield or barrier;
flexible light emitting circuitry positioned between said layer of flexible material and said transparent shield or barrier;
a shape retainer comprising first and second end regions which extend along the first and second ends of the pad, and first and second mid-regions which extend along the lateral edges of the pad, said first and second end regions and said first and second mid-regions comprising plastically deformable shape retaining material; and
a power source selected from; a power cord and a rechargeable battery;
wherein:
forming the light therapy pad into a curved shape causes plastic deformation of one or more regions of the shape retainer, which thereafter causes the light therapy pad to retain that curved shape; and
the light therapy pad is positionable, while being retained in said curved shape, in juxtaposition to said treatment area such that light emitted from the flexible light emitting circuitry passes through the translucent front shield or barrier and onto said treatment area.

22. A device according to claim 21 wherein the first and second end regions and the first and second mid-regions of the shape retainer comprise malleable metal.

23. A device according to claim 21 wherein the first and second end regions and the first and second mid-regions of the shape retainer comprise shape retaining plastic.

24. A device according to claim 21 wherein the flexible light emitting circuitry comprises light emitting diodes.

25. A device according to claim 24 wherein at least some of the light emitting diodes emit blue light.

26. A device according to claim 24 wherein at least some of the light emitting diodes emit red light.

27. A device according to claim 24 wherein at least some of the light emitting diodes emit infrared light.

28. A device according to claim 24 wherein the flexible light emitting circuitry comprises blue, red and near infrared light emitters.

29. A device according to claim 28 further comprising a controller useable for causing the device to alternately operate in in a plurality of light treatment modes.

30. A device according to claim 32 wherein the plurality of light treatment modes comprises:
a first mode wherein primarily or entirely blue light is emitted;
a second mode wherein primarily or entirely red light is emitted; and
a third mode wherein primarily or entirely near infrared light is emitted.

31. A device according to claim 21 wherein the power source comprises a rechargeable battery and, when operating solely on power provided by the rechargeable battery, the device delivers from 2 to 10 Joules per square centimeter of light energy to the treatment area.

32. A device according to claim 31 wherein the size of the treatment area is more than 700 square centimeters.

33. A device according to claim 32 wherein the device delivers said 2 to 10 Joules per square centimeter of light energy to a depth of 25 mm below a skin surface covering said treatment area.

34. A method for using a device according to claim 21, said method comprising the steps of:
forming the device into said curved shape thereby causing plastic deformation of said one or more regions of the shape retainer, which thereafter causes the light therapy pad to retain that curved shape;
positioning the transparent shield or barrier in juxtaposition to said treatment area; and
operating the device to cause the flexible light emitting circuitry to emit light which passes through the translucent material of the front shield or barrier and onto said treatment area.

35. A method according to claim 34 wherein the power source comprises a rechargeable battery and wherein the device is operated solely on power provided by the rechargeable battery.

36. A method according to claim 35 wherein the device delivers from 2 to 10 Joules per square centimeter of light energy to a depth 25 mm below the skin surface covering said treatment area.
